(19) United States
(12) Patent Application Publication
(10) Pub. No.: US 2025/0255983 A1
(43) Pub. Date: Aug. 14, 2025

(54) CRIPTO-POSITIVE LIPID VESICLES FOR USE IN THE THERAPEUTIC TREATMENT OF AGGRESSIVE TUMOURS

(71) Applicant: CONSIGLIO NAZIONALE DELLE RICERCHE, Roma (IT)

(72) Inventors: Giovanna Lucia LIGUORI, Caserta (IT); Francesca MANTILE, San Giorgio a Cremano (Napoli) (IT)

(21) Appl. No.: 18/856,706

(22) PCT Filed: Apr. 12, 2023
(86) PCT No.: PCT/IB2023/053735

§ 371 Date: Oct. 14, 2024

(30) Foreign Application Priority Data
Apr. 15, 2022 (IT) 102022000007580

(51) Int. Cl. A61K 47/69; A61K 31/337; A61K 31/4745; A61K 31/475; A61K 31/495; A61K 31/7048; A61K 33/243; A61K 35/13; A61K 47/64

(52) U.S. Cl. CPC A61K 47/6911; A61K 31/337; A61K 31/4745; A61K 31/475; A61K 31/495; A61K 31/7048; A61K 33/243; A61K 35/13; A61K 47/64

(57) ABSTRACT

A method of inhibiting or reducing the migratory ability of tumour cells in a subject is provided. The method involves administering to the subject a composition including Cripto-positive lipid vesicles, the Cripto protein being displayed on the surface of the Cripto-positive lipid vesicles.

The present invention falls within the field of oncology and, more particularly, concerns the treatment of highly infiltrating and/or metastasizing aggressive tumours, such as for example glioblastoma.

The ability of cancer cells to migrate, the degree of tumour infiltration, and the formation of metastases are key aspects in the prognosis of patients with cancer disease. In this context, glioblastoma (GBM) is the most common malignant tumour among brain neoplasms, with an annual incidence of about 4-5 cases per 100,000 people, predominantly men between the ages of 55 and 75, with higher incidence in the Caucasian population than in African or Asian populations. The high aggressiveness and malignancy that characterize glioblastoma make it a tumour that is difficult to treat.

The most commonly used therapy for GBM is surgical removal of the tumour mass, followed by radiotherapy and chemotherapy. Surgical removal of GBM is quite complex due to the high number of cells that make up the tumour and their very high ability to infiltrate the surrounding healthy tissues, both of which are causes of high recurrence rates, and is highly dependent on the location and accessibility of the tumour mass. Where possible, extensive surgical therapy is crucial to improve patient prognosis and decrease the risk of recurrence. After surgical resection, and if this is not possible, patients undergo radiotherapy often associated with the use of chemotherapy. In this context, the drug of choice is Temozolomide, a DNA alkylating agent.

The median survival of glioblastoma patients is short. In the absence of treatment, it is at most 4 months after diagnosis, otherwise about 15 months, while less than 5% survive more than 5 years. In childhood, although rare, GBM is one of the groups of malignancies with the worst prognosis. Furthermore, depending on its location and due to increased intracranial pressure, the tumour mass may cause neurological symptoms such as headache, nausea, vomiting, dizziness, weakness in a part of the body, up to changes in the faculty of thought and behaviour, seizures, psychosis, hemiparesis, seriously compromising the patient's quality of life. To further complicate the situation, GBM develops high resistance to commonly used chemotherapeutics, while surrounding brain tissues are highly sensitive to adjuvant radiotherapy. The presence of the blood-brain barrier makes it more difficult for therapeutic molecules to reach the tumour cells.

Since GBM is a highly vascular tumour, early clinical trials focused on the development of inhibitors of the angiogenic factor (VEGF), such as Bevacizumab, without however providing stable results. Systematic genomic analysis of GBMs also identified mutations in genes encoding tyrosine kinase receptors (such as EGFR, PDGFR, VEGFR, MET, FGFGR) in about 95% of cases, therefore many inhibitors of these receptors were developed and then tested in clinical trials, even in combination with each other, without however giving the desired results. Immunotherapeutic approaches aimed at stimulating the anti-cancer immune response were also developed, using vaccines, oncolytic viruses, immunological checkpoint inhibitors, alone or in combination with classical chemotherapeutic therapies. However, the extremely immunosuppressive tumour microenvironment of GBM, GBM heterogeneity, and the difficulty of reaching its site undermine the effectiveness of these therapeutic approaches. Hence the urgency to find new molecules effective in counteracting the development of GBM and in particular the migratory ability of GBM cells, which is responsible for the high infiltration ability and aggressiveness of this type of tumour, together with new vectors for the delivery of molecules capable of efficiently crossing the blood-brain barrier.

Pilgaard, Linda et al. “Cripto-1 expression in glioblastoma multiforme.” Brain pathology (Zurich, Switzerland) vol. 24,4 (2014): 360-70. doi: 10.1111/bpa.12131 describes a study performed on patients with glioblastoma multiforme (GBM), in which elevated plasma levels of the CR-1 (Cripto-1) protein were found to correlate significantly with a shorter overall survival. CR-1 targeted therapies are also considered to be used to treat GBM patients with CR-1-positive profiles. However, Pilgaard, Linda et al., 2014 does not provide any experimental, even preliminary, evidence to support the effectiveness of the hypothesized anti-CR-1 strategies, nor does it provide any suggestions as to the possible therapeutic agents by which such strategies could be implemented.

There is therefore a need to provide a therapeutic agent which is effective against aggressive tumours, i.e., highly infiltrating and/or metastatic tumours, such as for example glioblastoma.

A further need is to provide a therapeutic agent which is capable of crossing the blood-brain barrier and thus capable of reaching brain tumours as well.

Yet another need is to provide a therapeutic agent which is capable of inhibiting or reducing the migration ability of tumour cells, counteracting the invasive capacity of the tumour and the formation of metastases.

Yet another need is to provide a therapeutic agent which is not prone to induce resistance to chemotherapy and is therefore suitable for use in the therapeutic treatment of chemotherapy-resistant tumours.

These and other needs are met by the present invention, which provides a composition comprising Cripto-positive lipid vesicles, wherein the Cripto protein is displayed on the surface of the lipid vesicles, for use in the therapeutic treatment of a tumour or in the therapeutic or preventive treatment of tumour metastases.

According to a preferred embodiment, the Cripto-positive lipid vesicles for use according to the invention are liposomes or extracellular vesicles (EVs).

Where the lipid vesicles for use according to the invention are liposomes, these may be conventional liposomes or suitably modified liposomes so as to enable them to cross the blood-brain barrier. Functionalized liposomes capable of crossing the blood-brain barrier and reaching brain tumours are described in the literature. For example, liposomes bearing the ApoE protein on their surface (Ouyang, J., Jiang, Y., Deng, C., Zhong, Z., & Lan, Q. (2021). Doxorubicin Delivered via ApoE-Directed Reduction-Sensitive Polymersomes Potently Inhibit Orthotopic Human Glioblastoma Xenografts in Nude Mice. International journal of nanomedicine, 16, 4105-4115), liposomes surface-functionalized with p-aminophenyl-α-D-manno-pyranoside (MAN) and wheat germ agglutinin (Kong, D., Hong, W., Yu, M., Li, Y., Zheng, Y., & Ying, X. (2022). Multifunctional Targeting Liposomes of Epirubicin Plus Resveratrol Improved Therapeutic Effect on Brain Gliomas. International journal of nanomedicine, 17, 1087-1110), and biomimetic liposomes (Li, J., Zeng, H., You, Y. et al. Active targeting of orthotopic glioma using biomimetic liposomes co-loaded elemene and cabazitaxel modified by transferritin. J Nanobiotechnol 19, 289 (2021)) have been described.

According to another preferred embodiment, the Cripto-positive lipid vesicles display the Cripto protein on their surface because they are EVs derived from tumour cells expressing the Cripto gene, such as for example human teratocarcinoma cells, human colon cancer cells, and others.

According to another preferred embodiment, the Cripto-positive lipid vesicles (conventional liposomes, functionalized liposomes as mentioned above, or EVs) display the Cripto protein on their surface because they have been engineered to bind the Cripto protein to the lipid membrane surrounding them, through interaction with the lipid layer as such or following appropriate lipid functionalization techniques (see e.g. the review Riaz M K, Riaz M A, Zhang X, et al. Surface Functionalization and Targeting Strategies of Liposomes in Solid Tumor Therapy: A Review. Int J Mol Sci. 2018; 19 (1): 195. Published 2018 Jan. 9. doi: 10.3390/ijms19010195).

Even when this is not expressly specified, the term “Cripto-positive lipid vesicles” herein always indicates that the Cripto protein is displayed on the surface of the vesicles.

Additional features and advantages of the invention are defined in the dependent claims, which form an integral part of the specification.

The Cripto protein present on the surface of the lipid vesicles used in the invention is known as Teratocarcinoma-Derived Growth Factor 1 (TDGF-1) or CR-1 (UniProtKB accession number P13385). The Cripto gene is known to be expressed at high levels by human teratocarcinoma cells, but has also been found to be expressed in many other human cancers, such as breast cancer, hepatocellular carcinoma, nasopharyngeal carcinoma, esophageal squamous cell carcinoma (ESCC), non-small cell lung cancer, clear cell renal cell carcinoma (ccRCC), bladder cancer, cervical cancer, ovarian cancer, uterine cancers, uveal melanoma, intraductal papillary mucinous neoplasms (IPMNs), prostate cancer, gastric and colorectal carcinomas, perihilar cholangiocarcinoma.

The present invention is based on studies carried out by the inventors—which will be described in detail below—which show that lipid vesicles displaying Cripto on their surface (in this case extracellular vesicles (EVs) isolated from human teratocarcinoma cells, a highly aggressive type of tumour) exhibit a significant inhibitory effect on the migration of cancer cells, counteracting their invasive capacity that leads to the formation of metastases. The inventors also verified that blocking the surface-displayed Cripto protein through a specific antibody reduces the anti-migration effect exerted by the Cripto-positive lipid vesicles on tumour cells. This result indicates that the Cripto protein displayed on the surface of lipid vesicles plays a significant role in reducing the migration activity of tumour cells. Since, as mentioned above, the Cripto protein is known to be present in a number of human tumours, in addition to teratocarcinoma, extracellular vesicles isolated from other types of tumour cells, other than teratocarcinoma, are also expected to contain the Cripto protein and have a similar effect on tumour cell migration. The human colon cancer cell line SW-620 (ATCC CCL-227™) is a non-limiting example thereof. Colon cancer is a tumour expressing the Cripto gene (Sato J, Karasawa H, Suzuki T, et al. The Function and Prognostic Significance of Cripto-1 in Colorectal Cancer. Cancer Invest. 2020;38(4):214-227. doi:10.1080/07357907.2020.1741604), and in particular the SW-620 cell line, which is a highly metastatic line (De Toledo et al., 2012; 10.1371/journal.pone.0048344), is among those with the highest expression levels of Cripto as reported by the Depmap cell line database (https://depmap.org/portal/). EVs (large and/or small) can be isolated from SW-620 cells using the dUC procedure (see below), tested for the presence of Cripto and then used in migration/wound healing assays on U87 cells as described below.

It is also to be expected that the anti-migratory effect of the Cripto-positive lipid vesicles for use according to the invention is not limited to glioblastoma alone, but extends to other tumours, such as the above-listed tumours expressing high levels of Cripto. It is also likely that cancer cells that do not over-express Cripto can still be responsive to its administration from the outside via lipid vesicles. In fact, the ability of lipid vesicles to deliver their contents to heterogeneous cells, for example different types of tumour cell lines, or tumour and normal cells, is well known (Mantile F., Franco P., Stoppelli M. P., Liguori G. L. Biological role and clinical relevance of extracellular vesicles as key mediators of cell communication in cancer. (2021). Doi: 10.1016/bs.abl.2020.05.006).

It should also be noted that, although the experiments on the anti-migratory effect were carried out with large EVs, it is likely that small EVs also provide similar results in terms of anti-migratory activity. The meaning of the terms “large EVs” and “small EVs” is as follows. As is known, EVs are extremely heterogeneous in size, content and density. EVs are conventionally classified by size and density. Based on their size, EVs are distinguished into small EVs (sEVs), whose diameter ranges from 20-30 nm to 200-250 nm, and large EVs (IEVs), whose diameter ranges from 200-250 nm to 1-2 μm. Slight variability in size may also depend on the measurement/observation technique used. Separation of EVs is performed by differential ultracentrifugation (dUC), which is the most conventional method of isolation of EVs and is still considered the gold standard, as evaluated by a worldwide ISEV survey (Gardiner C, Di Vizio D. Sahoo S, et al. Techniques used for the isolation and characterization of extracellular vesicles: results of a worldwide survey. J Extracell Vesicles. 2016;5:32945). The dUC technique involves running multiple centrifugation cycles at increasing speed. By centrifuging at low speed, i.e., 300×g and then 2,000×g. the biofluids are first deprived of cells and cellular debris, respectively. Large EVs have been shown to settle at a rate of approximately 10,000×g. whereas small EVs conventionally settle at 100,000×g by high-speed ultracentrifugation. EVs settling at 100,000×g and 10,000×g are defined as small EVs and large EVs, respectively (Yekula et al., 2020, doi:10.1080/20013078.2019.1689784).

The anti-tumour effect of the Cripto-positive lipid vesicles observed by the present inventors is absolutely surprising, since, in the state of the art, the Cripto protein is described as a factor involved in tumour progression, being able to stimulate tumour proliferation, migration and angiogenesis. In this regard, see for example the aforementioned article Pilgaard L. et al., 2014, and Bianco, Caterina et al. “Role of Cripto-1 in stem cell maintenance and malignant progression.” The American journal of pathology vol. 177,2 (2010): 532-40. doi:10.2353/ajpath.2010.100102.

As regards the role of extracellular vesicles in cancer therapy, reference is made to the review Tickner, Jacob A et al. “Functions and therapeutic roles of exosomes in cancer.” Frontiers in oncology vol. 4 127. 27 May 2014, doi: 10.3389/fonc.2014.00127, which reports that exosomes secreted by tumour cells, through their interaction with a wide range of tissues, are typically capable of generating a pro-tumour microenvironment essential for carcinogenesis, as well as contributing to the development of resistance to chemotherapeutics. Tickner, Jacob A et al., 2014 also mentions some possible cancer treatment approaches involving exosomes. However, these approaches consist in the use of exosomes as carriers for therapeutic agents, such as chemotherapeutic drugs, microRNAs and siRNAs capable of regulating gene expression, and tumour antigens capable of eliciting an immune response against the tumour, or they consist in the removal of exosomes from the circulatory system as a therapeutic option for mitigating the metastatic effect of the exosomes per se.

Patent application EP 3 436 056 A describes the use of tumour-derived exosomes in the treatment of cancer, specifically for breast cancer. However, EP 3 436 056 A relates to vesicles originating from low metastatic breast cancer cell lines, whose inhibitory effect on metastases is associated with the intracellular factor NFTATC4, a factor that was previously known to inhibit cell motility in breast cancer (Fougère, M et al. “NFAT3 transcription factor inhibits breast cancer cell motility by targeting the Lipocalin 2 gene.” Oncogene vol. 29,15 (2010): 2292-301. doi:10.1038/onc.2009.499).

The unexpected results obtained by the inventors provide for a composition with anti-cancer activity which, more particularly, is advantageously capable of inhibiting or reducing the migratory activity of cells from aggressive tumours, i.e., highly infiltrating and/or metastatic tumours, thereby inhibiting or reducing the ability to form metastases. Furthermore, because it consists of lipid vesicles (optionally functionalized EVs or liposomes), which are known to be molecule carriers, the composition of the invention is advantageously capable of crossing the blood-brain barrier, thus reaching brain tumours as well. A further advantageous characteristic is that, in the studies carried out by the inventors, the Cripto-positive lipid vesicles have shown that they do not increase the viability of glioblastoma cells, either in the absence or in the presence of Temozolomide (TMZ), the latter data possibly indicating the absence of chemoresistance induction.

Due to their ability to act as molecule carriers, the Cripto-positive lipid vesicles used in the invention are also suitable to be loaded with an anti-cancer agent selected according to the tumour to be treated. Examples of anti-cancer agents to be used for this purpose are Temozolomide, cisplatin, or naturally occurring compounds such as etoposide, used to treat GBM. Other interesting natural compounds to be tested are vinca alkaloids (vincristine vinblastine), used for the treatment of testicular and breast cancer, camptothecin analogues (topotecan and irinotecan) used to counteract cervix, ovary and small cell lung cancer, taxanes (Paclitaxel) used against various cancers such as ovary, breast and non-small cell lung cancer, and semi-synthetic analogues thereof (Docetaxel), also used in the treatment of stomach adenocarcinoma, prostate cancer and some head and neck cancers.

Alternatively, the combination therapy with Cripto-positive lipid vesicles and anti-cancer agent may be administered as a combined preparation, or kit-of-parts. Accordingly, another aspect of the invention is a combined preparation, or kit of parts, comprising the composition of Cripto-positive lipid vesicles as defined above and an anti-cancer agent as defined above, for simultaneous, separate or sequential use in the therapeutic treatment of a tumour or in the therapeutic or preventive treatment of tumour metastases. Non-limiting examples of tumours suitable to be treated with the Cripto-positive lipid vesicles, optionally in combination with an anti-cancer agent, are glioblastoma, breast cancer, hepatocellular carcinoma, nasopharyngeal carcinoma, esophageal squamous cell carcinoma (ESCC), non-small cell lung cancer, clear cell renal cell carcinoma (ccRCC) bladder cancer, cervical cancer, ovarian cancer, uterine cancers, uveal melanoma, intraductal papillary mucinous neoplasms (IPMNs), prostate cancer, gastric and colorectal carcinomas, perihilar cholangiocarcinoma.

The following examples describe the experiments carried out by the present inventors and are provided for illustration purposes only and should not be construed as limiting the scope of the invention. The examples refer to the accompanying figures, in which:

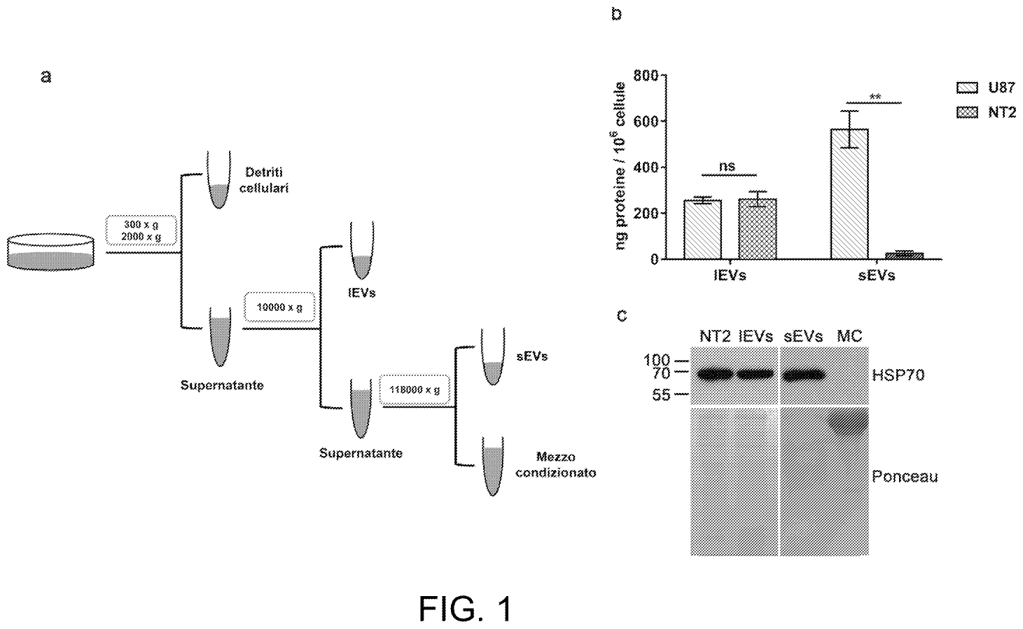

FIG. 1 relates to the isolation of EVs from Ntera2 human teratocarcinoma cells. (a) General outline of the protocol used for EV purification. (b) Comparison of EVs produced by U87 GBM cell line (n=3) and Ntera2 cell line (NT2) (n=6). The figure shows that NT2 cell line produces a similar amount of IEVs (also referred to as “microvesicles” or large EVs) compared to U87, but significantly lower amounts of sEVs (also referred to as “nanovesicles” or small EVs). Data are shown as mean±S.E.M. **p<0.01. (c) Western blot. Equal amounts (20 μg) of cell lysate (NT2), IEVs, sEVs and conditioned medium (CM) were immunoblotted with antibodies against the EV marker Heat Shock Protein 70 kilodaltons (HSP70). The lower part shows the blot stained with Ponceau Red to highlight the presence of proteins.

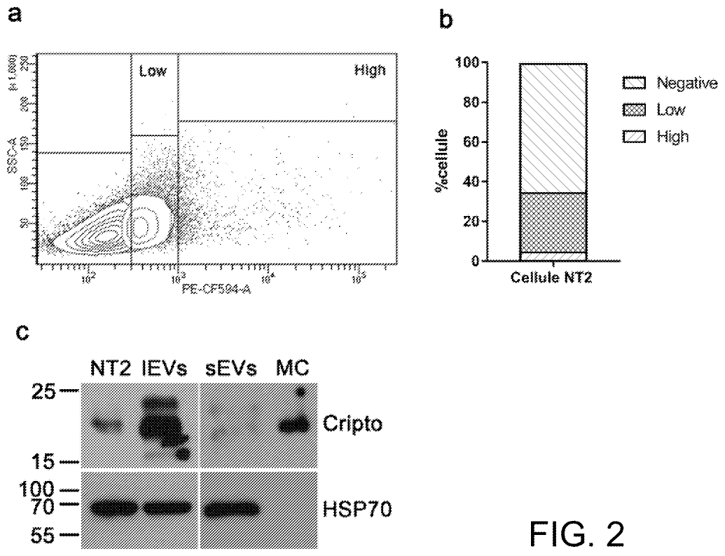

FIG. 2 relates to the results from Cripto expression studies in Ntera2 cells and EVs. (a, b) Fluorescence Activated Cell Sorter (FACS) analysis identifying two different populations of Ntera2 (NT2) (Low and High) regarding the presence of Cripto on the membrane. Altogether, approximately 40% of cells display Cripto on their surface. (c) Western blot. Equal amounts (20 μg) of Ntera2 cell lysate (NT2), IEVs, sEVs and conditioned medium (CM) were immunoblotted with antibodies against CRIPTO and the EV marker HSP70. The figure shows that IEVs are particularly enriched in Cripto compared to sEVs and cell lysates.

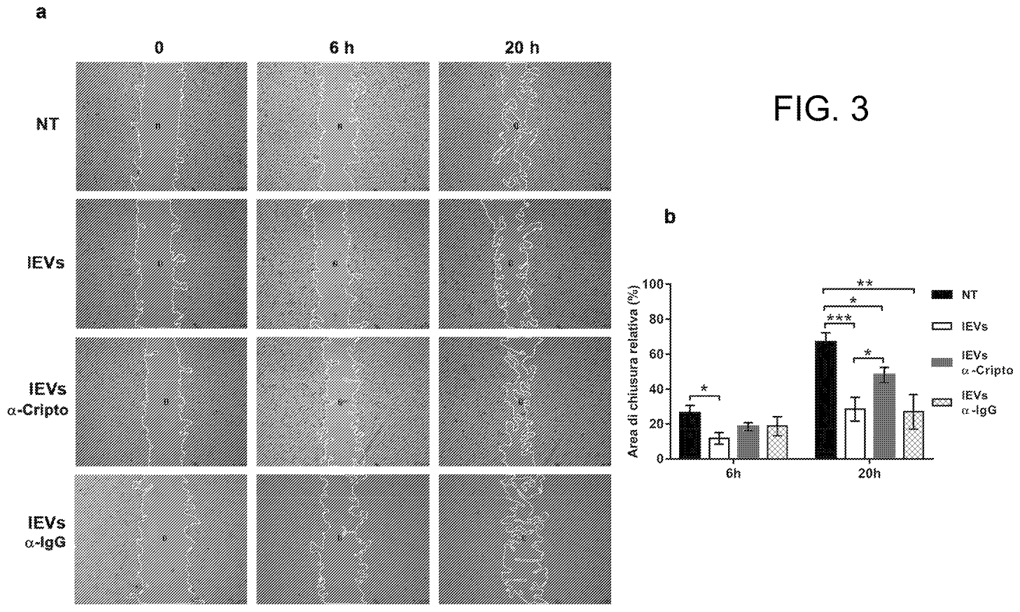

FIG. 3 relates to the results from U87 GBM tumour cell migration inhibition studies by means of the wound healing assay. (a) Cell migration at 0, 6 and 20 hours. Non-treated (NT) U87; U87 treated with microvesicles (IEVs) from Ntera2 cells; U87 treated with microvesicles from Ntera2 cells previously incubated with anti-Cripto antibody (IEVs-α-Cripto); U87 treated with microvesicles from Ntera2 cells previously incubated with non-specific antibody (1EVs-α-IgG). The solid line defines the open area, the extent of which is measured by the ImageJ software. (b) The values refer to three independent experiments, each in duplicate. Data are shown as mean±S.E.M. The figure shows that microvesicles (IEVs) from Ntera2 cells significantly reduce the closure rate and thus the migration of U87 cells already at 6 hours of treatment. The effect is most visible at 20 hours. Pre-treatment of microvesicles with a Cripto-specific antibody, but not with the non-specific antibody (IEVs-α-IgG), reduces the anti-migratory activity after 20 hours of treatment. *p<0.05; **p<0.01; ***p<0.001.

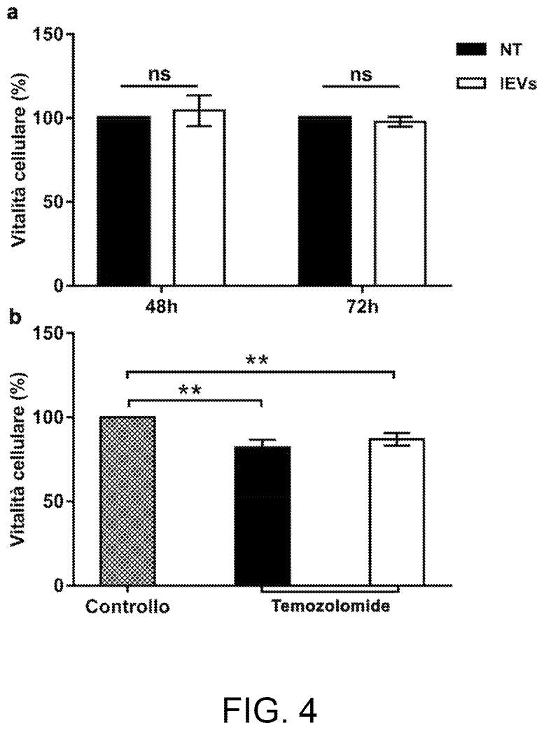

FIG. 4 relates to the effect of microvesicles from Ntera2 on the viability of U87 cells, in the presence and absence of chemotherapy. (a) U87 cells were treated with 10 μg/mL Ntera2-IEVs for 48 or 72 hours and viability was measured by MTT assay. Non-treated cells (NT) were used as controls. (b) Temozolomide was added to the indicated samples and MTT assay was performed after 72 hours of incubation. The results are shown as mean+/−S.E.M. from two independent experiments, each in duplicate. **p<0.01. The figure shows that microvesicles from Ntera2 have no significant effect on the viability of U87 cells, either in the presence or in the absence of chemotherapy.

### EXAMPLES

### Example 1: Ntera2 Cell Line Culture, and EV Isolation and Characterization

Ntera2 cells were purchased from ATCC (ATCC-CRL-1973), amplified and cultured in DMEM supplemented with 10% Fetal Bovine Serum (FBS), penicillin (100 U/mL) and streptomycin (100 mg/mL), GIBCO, at 37° C. in an atmosphere of 5% CO₂. For EV purification, Ntera2 cells were cultured in 150 mm plates (Corning, 430599) at a density of approximately 25,000 cells/cm² and cultured under standard conditions for 24 hours. The following day, cells were washed twice with PBS and cultured for 48 hours in DMEM supplemented with 10% EV-depleted Fetal Bovine Serum (FBS). The conditioned medium was collected after 48 hours from 15 culture plates and the EVs were isolated by differential centrifugation. In short, the conditioned media were centrifuged twice at 300×g. 4° C. for 10 minutes to remove cell debris. The supernatant fractions were further centrifuged twice at 2000×g. 4° C. for 10 minutes. Large EVs (IEVs), also referred to as microvesicles, were spun down by centrifugation at 10,000 g. 4° C. for 30 minutes, followed by PBS wash. Small EVs (sEVs), also referred to as nanovescicles, were spun down by ultracentrifugation at 118,000×g. 4° C. for 70 min, followed by PBS wash. The IEV and sEV fractions thus sedimented were then dried and resuspended in PBS. EVs (IEVs and sEVs) were analysed using the BCA Protein Assay Kit (Thermo Fishers Scientific) to measure their protein concentration, under an electron microscope (Cryo-EM and SEM) to show their morphology, and by Western Blot to test for the presence of specific markers. The Ntera2 cell line and the U87 cell line were shown to produce similar amounts of IEVs (microvesicles), but the Ntera2 cell line was shown to produce significantly lower amounts of SEVs (nanovesicles) than the U87 line (see, in particular, FIG. 1b). The presence of the EV marker HSP70 was also assessed, both in the cell lysate and in the fractions obtained by differential centrifugation of the conditioned medium. The conditioned medium (CM) depleted of the vesicles as a result of dUC, as expected, is devoid of it, confirming successful separation (see, in particular, FIG. 1c).

### Example 2: Cripto Expression in Ntera2 Cells and EVs

### Western Blot

The protein content of the EVs was measured using the BCA Protein Assay Kit (Thermo Fishers Scientific). Cells were lysed with RIPA buffer (50 mM Tris-HCl pH 8, 150 mM NaCl, 1 mM EDTA pH 8, 1% Triton X-100) supplemented with Complete Protease Inhibitor Mixture tablets (Roche Diagnostics). Samples in reducing Laemmli buffer were boiled at 95-100° C. for 5 minutes, loaded and separated by 12% Sodium Dodecyl Sulfate-Polyacrylamide Gel Electrophoresis (SDS-PAGE). Then, the proteins were transferred onto PVDF membranes, and the membranes were incubated with a 5% milk-TBS-T solution for 1 hour at room temperature, followed by overnight incubation at 4° C. with the primary antibody. Anti-Cripto (1:500 dilution, Abcam, 133236) and anti-HSP70 (1:500 dilution, Santa Cruz Biotechnology, clone W27) were used as the primary antibodies. The membranes were then incubated for 1 hour at room temperature with a 1:10,000 dilution of secondary antibody (SIGMA, 12-348). The membranes were then washed three times in TBS-T buffer and chemiluminescence was detected using an enhanced chemiluminescence kit (Clarity Western ECL substrate, Biorad, 1705060) according to the manufacturer's protocol.

### Flow Cytometry (FACS)

Ntera2 cells were detached using 0.5% trypsin, centrifuged at 300×g and resuspended in PBS 5% FBS. 10⁶ cells were resuspended in a volume of 100 μl and incubated with the anti-Cripto primary antibody (1:100 dilution, Abcam ab 19917) for 1 hour on ice in the dark. After two PBS washes, cells were incubated with Alexa Fluor 594 donkey anti-rabbit secondary antibody (1:100 dilution, Invitrogen A21207) for 1 hour on ice in the dark. After two PBS washes, cells were resuspended in PBS 5% FBS, filtered and analysed with BD FACS ARIAIII flow cytometer, and data was collected using the BD FACSDiva software. For each sample, data was collected on 20,000 cells.

FACS analysis showed two different Ntera2 populations (Low and High) as regards the presence of the Cripto protein on the membrane; overall, approximately 40% of Ntera2 cells were shown to display Cripto on their surface (FIGS. 2a and 2b). Western blot analysis showed that IEVs purified from Ntera2 cells are particularly enriched in Cripto compared to sEVs and cell lysates.

### Example 3: U87 Cell Migration Assay

U87 GBM cells (Merck 89081402) were amplified and cultured in DMEM supplemented with 10% Fetal Bovine Serum (FBS), penicillin (100 U/mL) and streptomycin (100 mg/mL), GIBCO, at 37° C. in an atmosphere of 5% CO₂. For the assay, the cells were plated in complete medium inside an Ibidi insert (Ibidi, GmbH, Martinsried, Germany, 81176) placed in a 24-well plate at a concentration of 13,000 cells/well. After 24 hours, the Ibidi insert was removed, the cells were washed with PBS, and the complete medium was replaced with DMEM, 2% EV-depleted FBS, 2 μg/mL mitomycin C to inhibit cell proliferation. Cells were treated with: 10 μg/mL Ntera2 IEVs; 10 μg/mL Ntera2 IEVs previously incubated for 1 hour at 4° C. with 1:100 anti-Cripto antibody-(Abcam, 19997) (IEVs-α-Cripto); 10 μg/mL Ntera2 1EVs previously incubated for 1 hour at 4°° C. with 1:100 HRP-conjugated anti-Rabbit IgG antibody (SIGMA, 12-348) (1EVs-α-IgG). Non-treated U87 cells were used as a control. Wound closure was observed under the Leica DMI 6000 inverted microscope equipped with a DFC420 digital camera at 5× magnification, after 6 and 20 hours, choosing a representative field for each well with the LASAF (Leica Application Software Advance Fluorescence) software. ImageJ software was used to analyse the results. The percentage of closure was calculated as follows: (initial area−final area)/initial area.

The migration and wound healing assay showed that microvesicles (IEVs) from Ntera2 cells significantly reduced the closure rate and thus the migration of U87 cells already at 6 hours of treatment and that the effect is most visible at 20 hours. Pre-treatment of microvesicles with a Cripto-specific antibody (IEVs-α-Cripto) reduced the anti-migratory activity after 20 hours of treatment. By contrast, pre-treatment of microvesicles with a non-specific antibody (IEVs-α-IgG) did not reduce their anti-migratory activity. These results are shown in FIGS. 3a and 3b.

### Example 4: Cell Viability Assay and Chemotherapy Sensitivity Test

U87 cells were plated in a 96-well plate in a volume of 100 μL (500 cells/well) in DMEM supplemented with 10% Fetal Bovine Serum (FBS), penicillin (100 U/mL) and streptomycin (100 mg/mL), GIBCO, at 37° C. in an atmosphere of 5% CO₂. After 24 hours, the culture medium was replaced with fresh medium supplemented with EV-depleted FBS enriched or not with 10 μg/mL of large EVs (IEVs) from Ntera2. In chemotherapy sensitivity experiments, 100 μM of freshly dissolved Temozolomide (TMZ) was added to the cells. After incubation at 37° C. for 24, 48 and 72 hours, 20 μL of CellTiter 96® AQueous One Solution Reagent (Promega) was added, and the plates were incubated at 37° C. for 2 hours. The reaction was stopped by adding 25 μL of 10% SDS, and the absorbance at 490 nm was quantified using a microplate spectrophotometer. The mean optical density (OD, absorbance) was used to calculate the percent cell viability as follows: percent cell viability=(OD treatment−OD blank-medium alone)/(OD non-treated control−OD blank)×100%.

The assays performed show that microvesicles from Ntera2 have no significant effect on the viability of U87 cells, either in the absence or in the presence of chemotherapy (FIGS. 4a and 4b). The use of specific antibodies against Cripto or of non-specific antibodies (see previous assay) also has no additional effect.

What is claimed is

1-18. (canceled)

19. A method of inhibiting or reducing the migratory ability of tumour cells in a subject in need thereof, the method comprising administering to the subject a composition comprising Cripto-positive lipid vesicles, wherein the Cripto protein is displayed on the surface of the Cripto-positive lipid vesicles.

20. The method of claim 19, wherein the subject has a Cripto-expressing tumour.

21. The method of claim 20, wherein the Cripto-expressing tumour is selected from the group consisting of glioblastoma, breast cancer, hepatocellular carcinoma, nasopharyngeal carcinoma, esophageal squamous cell carcinoma (ESCC), non-small cell lung cancer, clear cell renal cell carcinoma (ccRCC) bladder cancer, cervical cancer, ovarian cancer, uterine cancers, uveal melanoma, intraductal papillary mucinous neoplasms (IPMNs), prostate cancer, gastric and colorectal carcinomas, and perihilar cholangiocarcinoma.

22. The method of claim 19, wherein the Cripto-positive lipid vesicles are extracellular vesicles (EVs) derived from tumour cells expressing the Cripto gene.

23. The method of claim 22, wherein the EVs are derived from human teratocarcinoma cells.

24. The method of claim 19, wherein the Cripto-positive lipid vesicles are loaded with an antitumour agent.

25. The method of claim 24, wherein the antitumour agent is selected from the group consisting of temozolomide, cisplatin, etoposide, vincristine, vinblastine, topotecan, irinotecan, paclitaxel, and docetaxel.

26. The method of claim 19, wherein the composition further comprises an antitumour agent.

27. The method of claim 26, wherein the antitumour agent is selected from the group consisting of temozolomide, cisplatin, etoposide, vincristine, vinblastine, topotecan, irinotecan, paclitaxel, and docetaxel.

28. The method of claim 19, further comprising simultaneously, separately or sequentially administering to the subject an antitumor agent.

29. The method of claim 28, wherein the antitumour agent is selected from the group consisting of temozolomide, cisplatin, etoposide, vincristine, vinblastine, topotecan, irinotecan, paclitaxel, and docetaxel.
